(19) United States
(12) Patent Application Publication
(10) Pub. No.: US 2016/0058965 A1
(43) Pub. Date: Mar. 3, 2016

(54) CANNULA DEVICE

(71) Applicant: ATOM MEDICAL CORPORATION, Tokyo (JP)

(72) Inventors: Kazuo MATSUBARA, Tokyo (JP); Terumi MATSUBARA, Tokyo (JP); Kenji KOBAYASHI, Saitama (JP); Shinichi KOBAYASHI, Saitama (JP)

(21) Appl. No.: 14/712,586

(22) Filed: May 14, 2015

(30) Foreign Application Priority Data
Aug. 28, 2014 (JP) 2014-174502

(51) Int. Cl. A61M 16/06

(52) U.S. Cl. CPC A61M 16/0666; A61M 16/0683

(57) ABSTRACT

A cannula device which supplies respiration gas to a patient provided with: a nasal cannula including nasal pipes inserted into nares of the patient; a pair of supplying tubes connected to both ends of the nasal cannula respectively; a bracket provided at at least one of the nasal cannula or the supplying tubes; and a locking part provided at the bracket, and detachably locking a fixture which attaches the nasal cannula on a head of the patient.

### RELATED APPLICATIONS

This application claims priority under 35 U.S.C. §119 to Japanese Patent Application No. 2014-174502 filed on Aug. 28, 2014, the entire content of which is hereby incorporated by reference.

### BACKGROUND OF THE INVENTION

1. Field of the Invention

The present invention relates to a cannula device for supplying respiration gas such as oxygen gas to a human body.

2. Description of the Related Art

Conventionally, artificial respirators sending respiration gas including specified quantity of oxygen to a respiratory tract of a patient, devices for oxygen inhalation therapy, and the like are known. The respiration gas sent from these devices is supplied to a human body through a cannula device.

In Japanese Unexamined Patent Application, First Publication No. 2003-38647 (hereinafter, “JP '647”) for example, the cannula device which is provided with a nasal cannula in which protruding parts (nasal pipes) which are inserted into nares are provided between a pair of end parts (introducing pipes) which are connected to tubes, and supplying tubes connected to both end parts of the nasal cannula is disclosed.

In this cannula device, by inserting the protruding parts (the nasal pipes) of the nasal cannula to nares of a patient and connecting the supplying tubes to the artificial respirator or the like, the respiration gas is supplied to the patient through the protruding parts (the nasal pipes). JP '647 describes a method for fixing the cannula so as not to move downward by extending upward the supplying tubes connected to both end parts of the nasal cannula and hanging them on the ears of the patient after inserting the protruding parts (the nasal pipes) of the nasal cannula into the nares of the patient.

### SUMMARY OF THE INVENTION

### Problems to be Solved by the Invention

However, by such fixing methods as described above, since the supplying tubes are connected to the artificial respirator or the like after hanging on the left and right ears from the nares of the patient, it is necessary that the supplying tubes are longer in comparison with a case in which the supplying tubes are connected directly to the artificial respirator or the like from the nares of the patient. Accordingly, temperature of the respiration gas passing through the supplying tubes is reduced.

Moreover, when it is fixed as above, the supplying tubes are largely in contact with a vicinity of cheeks and a chin in a state in which the supplying tubes are held between the ears and a brain-pan of the patient, so that dew condensation is generated by temperature difference between body temperature of the patient and an inside of the supplying tubes.

In this case, it is possible to cover the supplying tubes by a heat insulator at a vicinity of the ears of the patient so that the supplying tubes are not directly and largely in contact with skin of the patient. However, it is uncomfortable if the heat insulator is used.

The present invention is achieved in consideration of the above circumferences, and has an object to provide a cannula device in which temperature reduction of respiration gas and generation of dew condensation can be prevented by shortening supplying tubes for the respiration gas.

### Means for Solving the Problem

A cannula device of the present invention, which supplies respiration gas to a patient, includes: a nasal cannula including nasal pipes inserted into nares of the patient; a pair of supplying tubes for the respiration gas connected to both ends of the nasal cannula respectively; a bracket provided at at least one of the nasal cannula or the supplying tubes; and a locking part provided at the bracket, and detachably locking a fixture which attaches the nasal cannula on a head of the patient.

In the cannula device according to the present invention, it is possible to fix the nasal cannula which is put on the nares on the head of the patient so as not to move downward by locking the fixture at the locking part of the bracket. Accordingly, it is not necessary to hang the supplying tubes on ears of the patient, so that lengths of the supplying tubes can be short. Furthermore, it is possible to reduce contacting parts of the supplying tubes which are in directly contact with skin of the patient.

In the cannula device of the present invention, it is preferable that the locking part of the bracket include at least one round-shaped open hole into which the fixture is inserted.

The cannula device of the present invention is effective for the fixture, for example, when a fine cord such as an elastic cord or the like is used. Particularly, when the elastic cord is used, if the open hole is formed at a slightly smaller diameter than that of the elastic cord, the elastic cord is held by the locking part only by inserting the elastic cord into the open hole without any work such as tying the cord or the like by a function of elasticity (contraction) of the elastic cord; as a result, the nasal cannula can be easily fixed on the head of the patient so as not to move downward.

In the cannula device of the present invention, it is preferable that the locking part of the bracket include at least one slit-shaped open hole into which the fixture is inserted.

In the cannula device of the present invention, since the open hole of the bracket is formed to have a slit-shape, it is possible to utilize a cord-like member such as a stretchable bandage, an elastic band or the like which is generally situated in a medical site for the fixture fixing the nasal cannula on the head of the patient.

By providing the plurality of open holes on the bracket, the plurality of fixtures having a cord-shape can be locked respectively. Since the nasal cannula is held by the fixtures at two parts, at an upper part and a lower part of the head of the patient, the nasal cannula can be more reliably fixed so as not to move downward.

In the cannula device of the present invention, it is preferable that a stopper ring bundling the supplying tubes be further provided movably along a longitudinal direction of the supplying tube.

In the cannula device of the present invention, the nasal cannula can be fixed on a chin of the patient by bundling and narrowing the supplying tubes below the chin by the stopper ring.

### Effects of the Invention

According to the cannula device of the present invention, since the supplying tubes can be fixed on the head instead of hanging on the ears, the supplying tubes can be shorten, so that the temperature reduction of the respiration gas flowing through the supplying tubes can be prevented. Moreover, by reducing the contacting parts of the supplying tubes which are in contact with the skin of the patient, it is possible to prevent the generation of the dew condensation owing to the temperature difference between the body temperature of the patient and inside temperature of the supplying tubes.

### BRIEF DESCRIPTION OF THE DRAWINGS

FIG. 1 is a front view showing an embodiment of a cannula device according to the present invention.

FIG. 2 is a top view of FIG. 1.

FIG. 3 is a left-side view of FIG. 1.

FIG. 4A is a schematic front view showing a bracket of the present embodiment.

FIG. 4B is a schematic side view showing the bracket of FIG. 4A.

FIG. 5A is a schematic front view showing another example of the bracket.

FIG. 5B is a schematic side view showing the bracket of FIG. 5A.

FIG. 6A is a schematic front view showing another example of the bracket.

FIG. 6B is a schematic side view showing the bracket of FIG. 6A.

FIG. 7A is a schematic top view showing a stopper ring.

FIG. 7B is a schematic front view showing the stopper ring of FIG. 7A.

FIG. 7C is a schematic side view showing the stopper ring of FIG. 7C.

FIG. 8 is a front view showing a state in which a cannula device is put on a human body.

### DETAILED DESCRIPTION OF THE PREFERRED EMBODIMENTS

Below, an embodiment of a cannula device of the present invention will be described referring to the drawings.

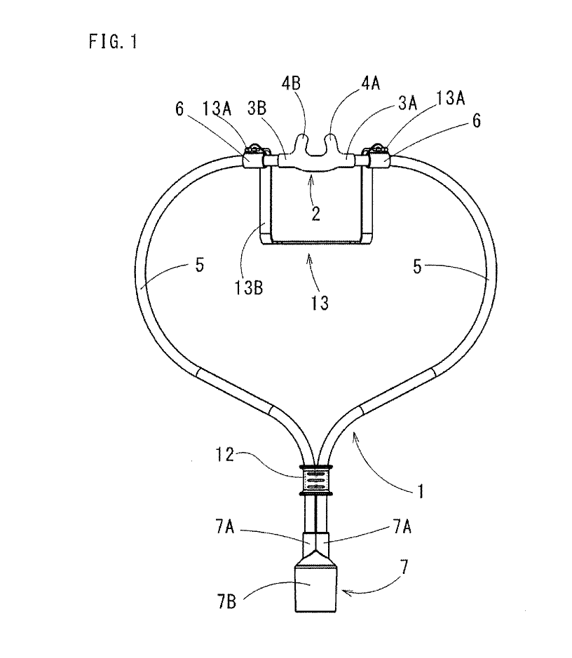

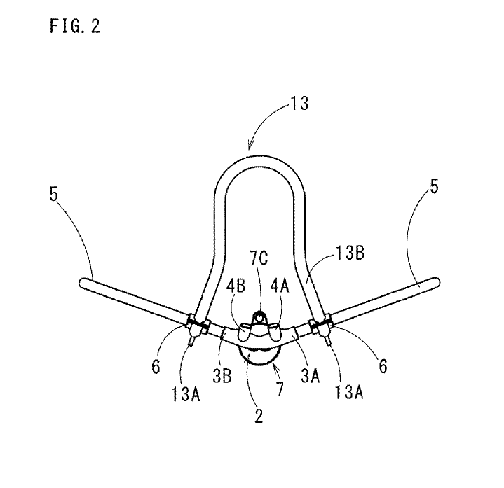

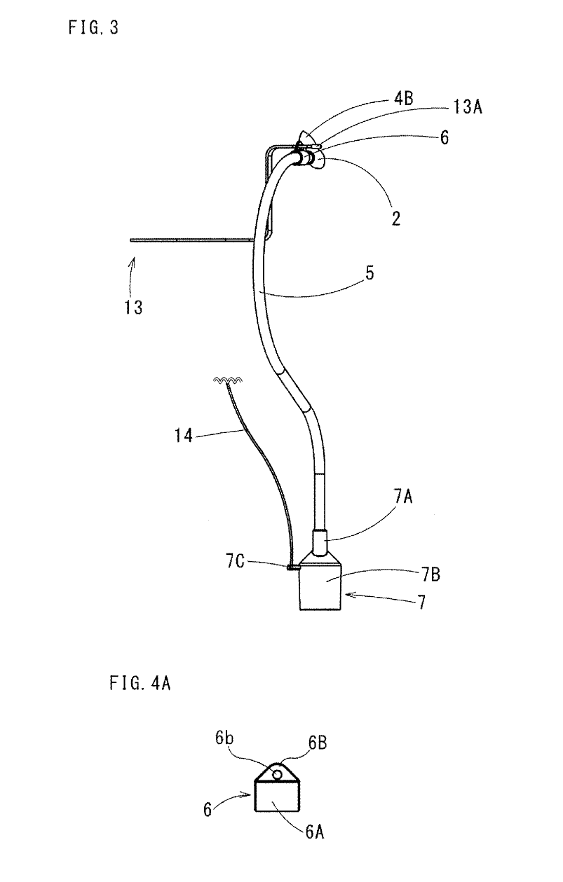

A cannula device 1 shown in FIG. 1 is formed from: a nasal cannula 2 in which nasal pipes 4A and 4B inserted into nares of the patient and introducing pipes 3A and 3B connected to supplying tubes 5 for respiration gas as stated below are respectively formed; a pair of the supplying tubes 5 for the respiration gas connected to the introducing pipes 3A and 3B of the nasal cannula 2; a pair of brackets 6 connected to the supplying tubes 5 respectively; a fixture 13 fixing the nasal cannula 2 on a head of a patient; a connector 7 connecting the supplying tubes 5 to an artificial respirator or the like (not illustrated); and a stopper ring 12 bundling the pair of supplying tubes 5.

The nasal cannula 2 is formed to be bent at a center part of a crosswise direction so as to extend along a surface of the face when it is put on a human body. The introducing pipes 3A and 3B are formed at both left and right end parts of the crosswise direction. Between these introducing pipes 3A and 3B, a pair of the nasal pipes 4A and 4B which are inserted into nares are provided upward in FIG. 1. The introducing pipes 3A and 3B are communicated with the nasal pipes 4A and 4B respectively. The nasal cannula 2 is entirely formed from soft synthetic resin such as polystyrene elastomer, silicone rubber, urethane, and the like.

The supplying tubes 5 are connected to the introducing pipes 3A and 3B of the nasal cannula 2 respectively, bundled by the connector 7 in one, and connected to the artificial respirator or the like through one connecting pipe (not illustrated). As the supplying tubes 5, both normal tubes for general-use and non-crash tubes in which ribs are formed on an inner surface of the tube along a longitudinal direction of the tube so as not to be clogged even if it is bent can be used. Although an external diameter of the supplying tubes 5 is not specifically limited; for example in the present embodiment, it is preferable to set the external diameter of the supplying tubes 5 to 5.5 mm.

The brackets 6 have cylindrical attachment parts 6A installed on the supplying tubes 5 respectively and locking parts 6B provided on outer peripheral surfaces of the attachment parts 6A respectively so as to fix the fixture 13. The attachment parts 6A are formed in a cylindrical shape and mounted on the supplying tubes 5 movably along the longitudinal direction. Although an inner diameter of the attachment parts 6A is not specifically limited, it is enough to set considering a relation with the external diameter of the supplying tubes 5; for example in the present embodiment, it is preferable to set the inner diameter of the attachment parts 6A to 5.8 mm.

The fixture 13 of the present embodiment is an elastic cord. Top-end parts 13A of the elastic cord are harden into thin-rod shapes; and a body part 13b is a band-shaped cord having elasticity.

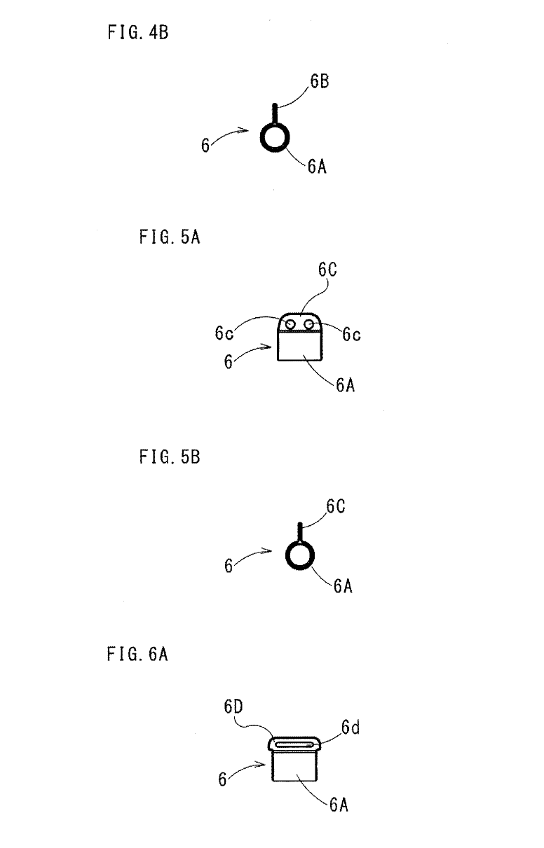

FIGS. 4A and 4B show a shape of the brackets 6 of the present embodiment; FIG. 4A is a front view and FIG. 4B is a side view.

The locking part 6B is formed of resin, rubber or the like harder than the fixture 13; and have a round-shaped open hole 6b having a diameter larger than that of the top-end parts 13A and smaller than that of the body part 13B of the aforementioned fixture 13. When the fixture 13 is inserted into the open hole 6b of the locking part 6B with a necessary length, the fixture 13 is pressed by the open hole 6b by the elasticity of the fixture 13, so that resistance power against moving along the longitudinal direction is generated on the fixture 13. Accordingly, the fixture 13 can be held by the locking part 6B without work of tying the fixture 13, so that the nasal cannula 2 does not move downward and can be easily fixed on the head of the patient.

Another example of the bracket 6 is shown in a front view by FIG. 5A and a side view by FIG. 5B.

In the bracket 6 shown in FIGS. 5A and 5B, the attachment part 6A is not modified from the above-mentioned present embodiment; though a locking part 6C provided on an outer peripheral surface of the attachment part 6A is formed to have round-shaped open holes 6c into which the fixture 13 can be inserted with intervals. A number is not limited if the open holes 6c are formed two or more; in this example, the two open holes are formed.

Since the plurality of open holes 6c are formed, it is easy to lock a plurality of the fixtures 13. By using the plurality of fixtures 13, the nasal cannula 2 can be fixed at two parts; at an upper part and a lower part of the head of the patient, so that the nasal cannula 2 can more reliably be fixed so as not to move downward. Moreover, one long fixture 13 can be used by extending between the two open holes 6c of the locking part 6C.

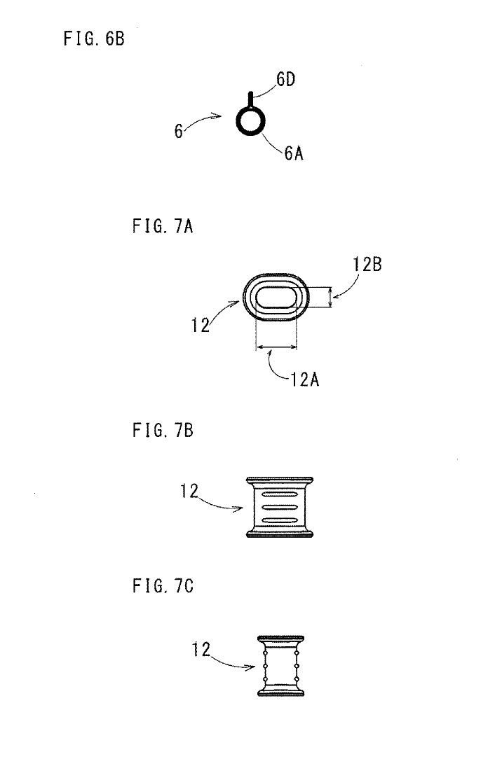

Moreover, another example of the bracket 6 is shown in a front view by FIG. 6A and a side view by FIG. 6B.

In the bracket 6 shown in FIGS. 6A and 6B, the attachment part 6A is not modified from the above-mentioned present embodiment; though a locking part 6D provided on the outer peripheral surface of the attachment part 6A is formed to have a slit-shaped open hole 6d into which the fixture 13 can be inserted. By this bracket 6, for example, the wide-width fixture 13 such as a stretchable bandage, an elastic band or the like which is generally situated in a medical site, can be used.

The connector 7 is integrally formed to have: a pair of insertion pipes 7A into which the two supplying tubes 5 are inserted respectively; a connecting port 7B communicating with the insertion pipes 7A; and a protruding part 7C in which a hole is formed for attaching a neck strap 14, so as to bundle the two supplying tubes 5 in one and connect them to the connecting pipe of the artificial respirator or the like. A specification of the connector 7 can be determined in accordance with a relation to the connecting pipe. Although it is not limited specifically, in the present embodiment for example, the connecting port 7B can be set to have a cylindrical shape with an outer diameter of 22 mm or 15 mm.

The two supplying tubes 5 are bundled in one and narrowed by being inserted into the stopper ring 12. The stopper ring 12 may be set to have a dimension in accordance with a relation with the external diameter of the supplying tubes 5 to be used; although it is not limited specifically, it is preferable that a width (at an inner dimension) 12A be set to 10.8 mm, and a thickness (at an inner dimension) 12B be set to 5.4 mm, in the present embodiment for example (refer to FIG. 7). It is preferable that the stopper ring 12 be made of soft material such as synthetic resin such as silicon, urethane or the like, elastomer or the like.

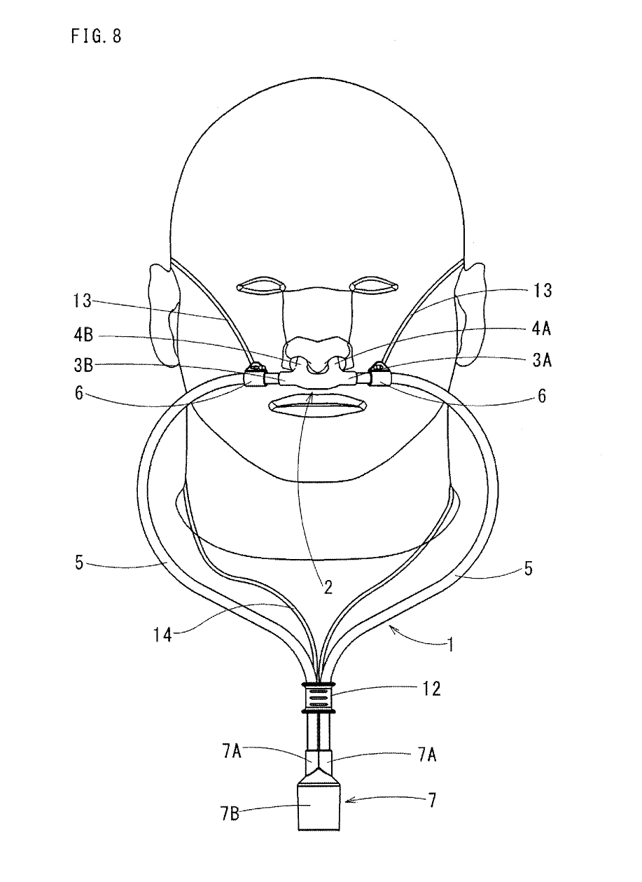

FIG. 8 shows a state in which the cannula device 1 of the present invention is put on a human body.

When using the cannula device 1 of the present invention: first, the neck strap 14 is put round a neck of a patient; the nasal pipes 4A and 4B are inserted into nares; and the nasal cannula 2 is fixed on a head of the patient by the fixture 13 locked on the bracket 6 so as not to move downward. Then, the cannula device 1 is connected to the artificial respirator or the like through the connector 7 and the connecting pipe; and fixed to a chin of the patient by bundling and narrowing the supplying tubes 5 below the chin by the stopper ring 12 as necessary.

The supplying tubes 5 for this cannula device 1 can be short since the supplying tubes 5 are not hanged on ears of the patient when the cannula device 1 is held on the head, so that the respiration gas can be prevented from being reduced of temperature in the supplying tubes 5. Furthermore, by reducing the contacting parts of the supplying tubes 5 which are in contact with the skin of the patient, it is possible to prevent the generation of the dew condensation owing to the temperature difference between the body temperature of the patient and inside temperature of the supplying tubes 5.

An embodiment of the present invention is described above. The present invention is not limited to the embodiment, and various modifications and revision may be made based on the scope of the present invention.

Although the attachment part 6A of the bracket 6 is formed cylindrically in the embodiment; it is not limited to the cylindrical shape, and it may have a ring-shape, or made of a C-shaped clip in which a part of a ring is cut off by a slightly smaller width than the external diameter of the supplying tubes 5.

The fixture 13 is not limited to the cord, the bandage, or the elastic band mentioned in the above embodiment; a net, a headset made of hard resin, metal or the like may be used for the fixture 13.

Although the bracket 6 is mounted on the supplying tubes 5 in the above embodiment, it is not limited to the supplying tubes 5; the bracket 6 may be mounted on the nasal cannula 2.

What is claimed is:

1. A cannula device which supplies respiration gas to a patient, comprising:
a nasal cannula including nasal pipes inserted into nares of the patient;
a pair of supplying tubes for the respiration gas connected to both ends of the nasal cannula respectively;
a bracket provided at at least one of the nasal cannula or the supplying tubes; and
a locking part provided at the bracket, and detachably locking a fixture which attaches the nasal cannula on a head of the patient.

2. The cannula device according to claim 1, wherein the locking part of the bracket includes at least one round-shaped open hole into which the fixture is inserted.

3. The cannula device according to claim 1, wherein the locking part of the bracket includes at least one slit-shaped open hole into which the fixture is inserted.

4. The cannula device according to claim 1, further comprising a stopper ring bundling the supplying tubes provided movably along a longitudinal direction of the supplying tubes.
